(19) United States
(12) Patent Application Publication
(10) Pub. No.: US 2021/0077667 A1
(43) Pub. Date: Mar. 18, 2021

(54) STENTS

(71) Applicant: University of Westminster, London (GB)

(72) Inventors: Ipsita Roy, Southampton Hampshire (GB); Rinat Nigmatullin, Bristol (GB); Pooja Basnett, Pinner Greater London (GB); Barbara Lukasiewicz, Compton Berkshire (GB)

(21) Appl. No.: 16/642,740

(22) PCT Filed: Aug. 30, 2018
(86) PCT No.: PCT/GB2018/052443

§ 371 Date: Feb. 27, 2020

(30) Foreign Application Priority Data
Aug. 31, 2017 (GB) 1713994.0
Sep. 14, 2017 (GB) 1714814.9

(51) Int. Cl. A61L 31/04; A61L 31/14; C08L 67/04; C08G 63/08; C08G 63/89; C12N 1/20

(52) U.S. Cl. CPC A61L 31/041; A61L 31/148; C08L 67/04; C08G 63/08; C12R 1/38; C12N 1/20; C08L 2205/025; C12N 2500/34; C12N 2500/36; C08G 63/89

(57) ABSTRACT

The invention provides bioresorbable polymeric stents made from polymer blends which include polyhydroxyalkanoates (PHAs). In particular, the invention provides stents having a stent body which comprises a polymer blend comprising: (a) from 5 to 40 wt. % of a first component which is a PHA copolymer comprising two or more different medium chain length hydroxyalkanoate monomer units; and (b) from 60 to 95 wt. % of a second component which is either a PHA homopolymer containing a short chain length hydroxyalkanoate monomer unit, or a polylactide (PLA). The invention further relates to polymer blends comprising (a) and (b).

### FIELD OF THE INVENTION

The present invention generally relates to stents, and more particularly to bioresorbable polymeric stents. These are made from polymer blends which include polyhydroxyalkanoates (PHAs) (herein referred to as “PHA blends”). The invention further relates to novel PHA blends and to methods for their preparation.

The invention further relates to certain novel PHA materials produced by the microorganism Pseudomonas mendocina CH50, to methods for the production of such PHA materials, and their use in the preparation of a PHA blend which may be used as a polymeric stent material.

### BACKGROUND OF THE INVENTION

Coronary artery disease (CAD) is responsible for a significant number of deaths in the developed world. Angioplasty procedures are widely used for the treatment of occluded arteries, but the problem of restenosis following the angioplasty treatment remains a significant problem. Restenosis is the closure of a peripheral or coronary artery following trauma to the artery caused by procedures used to open an occluded portion of the artery by angioplasty, for example, balloon dilation or laser ablation treatment of the artery. One aspect of restenosis may be simply mechanical, i.e. elastic rebound of the arterial wall following the angioplasty procedure. Such mechanical problems have been addressed by the use of stents which are placed in the narrowed artery during angioplasty to widen, provide support and maintain normal blood flow. Stents are typically inserted by means of a catheter into the vascular lumen and expanded into contact with the arterial wall to provide internal support, for example by balloon expansion.

Metal stents and polymer coated metal stents are widely used in angioplasty procedures. These are used not only as mechanical support, but may also be used as vehicles for the delivery of drugs to the injured arterial wall. For example, a drug-eluting stent may be manufactured by coating the surface of a metallic stent with an active drug, or by inclusion of an active drug within a polymeric coating. Both metal and polymer coated metal stents, however, suffer from the drawback that they are not biodegradable and so typically may need to be removed once treatment is complete. This involves a surgical procedure which can often lead to damage to the arterial wall and, in some cases, further restenosis problems.

The biodegradable stent has emerged as a potential solution to the various limitations of metal and polymer coated metal stents. These can completely degrade within the body after the clinical need for them has ended. However, to date, their clinical use has been limited due to poor mechanical properties of the polymer materials (e.g. these fail to provide adequate support to the injured artery) and, in some cases, due to the release of non-physiological (e.g. highly acidic) degradation products which can give rise to an inflammatory response.

ABSORB™ BVS, produced by Abbott, is a commercially available biodegradable drug-eluting stent. It consists of an out-of-phase zig-zag scaffold which is made of poly-L-lactic acid (PLLA) having an antimitotic drug-releasing poly-D-L-lactic acid (PDLLA) coating. The ABSORB™ BVS stent, however, still has some limitations, most notably in respect of its mechanical properties. For example, the high glass transition temperature (60-65° C.) of PLLA causes the stent to be brittle at body temperature which can result in fracture during insertion or during expansion. It also has poor mechanical strength due, at least in part, to the low orientation of the polymer chains, and the surface of the stent is unsuitable for the promotion of endothelialisation since PLLA and PDLLA polymers do not adequately support endothelial cell attachment and proliferation.

Coronary stents must be mechanically robust to withstand radial compressive forces imposed on the stent as it supports the arterial walls during use. In addition, a stent must possess sufficient flexibility to enable it to be crimped onto a deflated balloon and then expanded in vivo. These requirements, in addition to the need for biodegradability, remain a challenge in developing alternative materials for use in the production of stents.

Polyhydroxyalkanoates (PHAs) are hydrophobic storage polymers which are polyesters of 3-, 4-, 5- and 6-hydroxyalkanoic acids produced by a variety of bacterial species from renewable carbon sources under nutrient-limiting conditions. They are biodegradable and biocompatible in nature. PHAs are attractive materials for biomedical applications because of their natural origin, enhanced biocompatibility, biodegradability, lack of cytotoxicity and ability to support cell growth and cell adhesion. Therefore, there has been a great interest in the commercial use of these biodegradable polyesters for industrial as well as biomedical applications. Depending on the total carbon chain length in the monomeric units, PHAs can be classified as short chain length (SCL) or medium chain length (MCL) PHAs. SCL-PHAs are brittle and have a high melting temperature and high crystallinity, whereas MCL-PHAs are elastomeric in nature and have a low melting temperature and low crystallinity.

Poly(3-hydroxybutyrate), commonly referred to as “P(3HB)”, is one of the most extensively studied SCL-PHAs. However, one of the major hindrances in the extensive commercial use of P(3HB) is its brittle and rigid nature owing to its high crystallinity. Poly(3-hydroxyoctanoate) or “P(3HO)” is one of the most extensively studied MCL-PHAs, but this similarly has not yet been commercially exploited due to its low melting temperature and tensile strength.

Depending on the nature of the application, desired properties can be achieved by making blends of PHAs and/or composites with organic and inorganic additives. Polymer blends may be miscible (homogeneous), immiscible (heterogeneous), or partially miscible (compatible) in nature. Most polymers are immiscible with other polymer materials and undergo phase separation when physically blended.

When producing any blend of two or more polymer materials, it can be difficult to predict the properties of the resulting blend due to the formation of multiphase polymer systems. The properties of such systems are determined not only by the constituent components but, to a large extent, by the morphology of the multiphase system. Therefore properties of any polymer blend made by physical blending are difficult to predict. The interaction between the components of the blend can lead to enhanced properties, but equally may lead to no change in properties or, in some cases, less desirable properties. Such properties can include mechanical, thermal and morphological properties.

In a previous study, P(3HB) and P(3HO) blends were investigated and characterised with respect to their mechanical, thermal, surface and microstructural properties (Basnett et al., Reactive & Functional Polymers 73: 1340-1348, 2013). P(3HB) was isolated from the Gram-positive bacterium Bacillus cereus SPV. P(3HO) was isolated from the Gram-negative bacterium Pseudomonas mendocina CH50. Degradation and biocompatibility studies were carried out on the blend films containing various ratios of P(3HB) and P(3HO). The production of these blends with tailored desirable properties was aimed at a variety of medical applications including coronary stent development. Increased Young's modulus, tensile strength, thermal stability and tailorable biodegradability as compared to pure P(3HO) was found. The results of the cytotoxicity assessment also demonstrated increased biocompatibility of the blends with human microvascular endothelial cells. However, the authors concluded that a further improvement in the mechanical properties of the blends was required for the development of biodegradable stents.

A need thus still exists for alternative polymer materials which are suitable for the production of stents, in particular such materials which combine properties such as high radial strength, high crush recovery (i.e. flexibility), expandability, and biodegradability.

### SUMMARY OF THE INVENTION

We have now found that stents having desirable properties and, in particular, desirable mechanical properties such as tensile strength, elastic modulus and elasticity, can be produced using certain polymer blends containing PHA materials (“PHA blends”). These blends include at least one PHA polymer which is a PHA copolymer comprising two or more different medium chain length hydroxyalkanoate (HA) repeating units. These particular PHA blends have not been previously produced, nor has their use in the production of stents been previously suggested.

In one aspect the invention relates to a stent having a stent body which comprises a polymer blend comprising:
(a) from 5 to 40 wt. % of a first component which is a PHA copolymer comprising two or more different medium chain length hydroxyalkanoate monomer units; and
(b) from 60 to 95 wt. % of a second component which is either a PHA homopolymer containing a short chain length hydroxyalkanoate monomer unit, or a polylactide (PLA).

In another aspect the invention relates to a method of producing a stent, said method comprising forming a stent body from a polymer blend which comprises:
(a) from 5 to 40 wt. % of a first component which is a PHA copolymer comprising two or more different medium chain length hydroxyalkanoate monomer units; and
(b) from 60 to 95 wt. % of a second component which is either a PHA homopolymer containing a short chain length hydroxyalkanoate monomer unit, or a polylactide (PLA)

In a further aspect the invention relates to a polymer blend comprising:
(a) from 5 to 40 wt. % of a first component which is a PHA copolymer comprising two or more different medium chain length hydroxyalkanoate monomer units; and
(b) from 60 to 95 wt. % of a second component which is either a PHA homopolymer containing a short chain length hydroxyalkanoate monomer unit, or a polylactide (PLA)

In a yet further aspect the invention relates to a method of producing a PHA copolymer, said method comprising the steps of:
(a) culturing Pseudomonas mendocina CH50 in a culture medium comprising a carbon source other than glucose;
(b) harvesting biomass from the culture medium;
(c) extracting PHA from the harvested biomass; and
(d) optionally purifying the crude PHA whereby to obtain a purified PHA.

In yet another aspect the invention provides a PHA copolymer obtained or obtainable by culturing Pseudomonas mendocina CH50 in the presence of a culture medium which comprises a carbon source other than glucose.

### DETAILED DESCRIPTION OF THE INVENTION

Stents comprising PHA blends are provided. In at least some aspects the PHA blends herein described have one or more properties, particularly mechanical properties, which are enhanced relative to the same properties of each of the individual blend components. In at least some embodiments, one or more properties, especially mechanical properties, of the PHA blends herein described are enhanced relative to the same properties of the major component of the blend, i.e. the second component (component (b)).

### Definitions

As used herein, the term “polymer” refers to a molecular chain of repeating units that may be linear or branched. It includes homopolymers and copolymers.

The term “homopolymer” as used herein refers to a polymer with a single repeating unit. The term “copolymer” as used herein refers to a polymer with at least two different repeating units.

As used herein, the term “monomer” means a repeating unit of a polymer. The term “comonomer” refers to one of a least two monomers that are present in a copolymer.

As used herein, the term “polyhydroxyalkanoate” (or “PHA”) refers to a biodegradable polyester which is synthesised by a microorganism.

The term “PHA homopolymer” as used herein refers to a polymer with a single hydroxyalkanoate (HA) repeating unit. The term “PHA copolymer” as used herein refers to a polymer comprising two or more different hydroxyalkanoate (HA) repeating units.

Reference herein to a “short chain length PHA” (or “SCL-PHA”) means a PHA having 3 to 5 carbon atoms in its repeating units. A “medium chain length PHA” (or “MCL-PHA”) means a PHA having more than 5 carbon atoms in its repeating units. The terms “short chain length hydroxyalkanoate monomer unit” and “medium chain length hydroxyalkanoate monomer unit” should be construed accordingly.

The term “poly(3-hydroxyalkanoate)” (or “P(3HA)”) as used herein refers to a polymer in which each repeating unit comprises three carbons in the backbone. Any remaining carbons are in the side-chain. For example, “poly(3-hydroxybutyrate)” (or “P(3HB)”) means a homopolymer comprising 3-hydroxybutyrate units in which three carbon atoms of each unit are present in the backbone and one carbon atom in each unit is in the side-chain. The term “P(3HA)” may be used generally to refer to a homopolymer or a copolymer.

As used herein, “polylactic acid” or “polylactide” means a homopolymer of lactic acid units. It may also be referred to as “PLA”. The term “PLLA” refers to poly-L-lactic acid in which each of the lactic acid units has the L-configuration.

As used herein, “glass transition temperature” (Tg) is the temperature at which the amorphous domains of a polymer change from a relatively brittle vitreous state to a solid deformable or ductile state. Tg thus corresponds to the temperature at which the onset of segmental motion in the polymer chains occurs.

As used herein, the “melting temperature” (Tm) of a polymer is the temperature at which the polymer changes from a solid to a liquid state, i.e. the peak temperature at which a semi-crystalline phase melts into an amorphous phase.

As used herein, the term “polymer blend” is generally used to refer to a physical combination of two or more polymer components as opposed to a chemical combination in which monomer units of a polymer are chemically linked. A “PHA blend” refers to a physical combination of different polymers, at least one of which is a PHA polymer.

An “immiscible blend” refers to a blend having composition-independent Tg(s) and Tm(s). The observed Tg(s) and Tm(s) of the immiscible blend are about the same as those of the individual components of the blend. The number of Tg(s) and Tm(s) of an immiscible blend is the same as the number of components of the blend, e.g. a two component PHA blend will exhibit two composition-independent Tg(s) and two composition-independent Tm(s).

As used herein, “tensile strength” is a measure of the capacity of a material to withstand a loading tending to elongate it. It is the maximum tensile stress which a material will withstand prior to fracture. The ultimate tensile strength is the maximum load applied during a test divided by the original cross-sectional area of the sample.

As used herein, any reference to “elasticity” or “Young's modulus” is a measure of the stiffness of a material. It is the ratio of a component of stress or force per unit area applied to a material divided by the strain along an axis of applied force which results from the applied force. The modulus or the stiffness is generally the initial slope of the stress-strain curve at low strain, i.e. in the linear region.

As used herein, “elongation” of a material or “elongation at break” is a measure of the elastomeric properties of a material and is the amount of increase in length resulting from, for example, the tension to break the material. Generally it is expressed as a percentage of the original length of the material.

Tensile strength, Young's modulus and elongation at break may, for example, be determined according to the procedure in the examples presented herein using a 5942 Testing Systems (Instron) equipped with 500 N load cell at room temperature (20 to 25° C.).

Unless otherwise specified, as used herein “molecular weight” refers to weight average molecular weight (Mw) measured by Gel Permeation Chromatography (GPC) with polystyrene standards using, for example, chloroform as both the eluent and diluent for the samples. Calibration curves for determining molecular weights can be generated using polystyrene molecular weight standards. Weight average molecular weight is the sum of the products of the molecular weight of each polymer fraction multiplied by its weight fraction. GPC analysis can provide weight average molecular weight (Mw) and polydispersity index (PDI). Molecular weight (Mw) may, for example, be determined according to the procedure in the examples presented herein using a PLgel 5 m MIXED-C (300×7.5 mm) column calibrated using narrow molecular weight polystyrene standards from 162 Da to 15,000 kDa.

As referred to herein, the polydispersity index (PDI) of a polymer is calculated by dividing the weight average molecular weight of the polymer by its number average molecular weight. The number average molecular weight can be measured using GPC, for example as herein described.

As used herein the term “biocompatible” refers to materials which are not toxic in vivo, and which do not elicit severe inflammatory or chronic responses in vivo. Degradation products (i.e. metabolites) of such materials should also be biocompatible. “Biodegradation” refers to the process of breakdown or dissolving of a material under physiological conditions, preferably in a period of less than 5 years, e.g. less than 2 years. It refers to a process which takes place in an animal, e.g. a human, and may occur by any suitable mechanism, such as hydrolysis. The biodegradation process via hydrolysis can occur with or without the presence of enzymes, such as lipases and other hydrolytic enzymes. The term “biodegradable”, when used in respect of any of the materials herein described, should be construed accordingly.

The first component of the PHA blends herein described is a PHA copolymer comprising two or more different medium chain length HA units. The PHA copolymer thus comprises at least two hydroxyalkanoate comonomers, namely a first hydroxyalkanoate comonomer and a second hydroxyalkanoate comonomer, wherein said first and said second comonomers are different to one another.

In one embodiment, the PHA copolymer may include two different types of hydroxyalkanoate repeating units (a “binary” PHA copolymer). In another embodiment, the PHA copolymer may further comprise a third hydroxyalkanoate comonomer in which each of said first, second and third comonomers is different from each other (a “ternary” PHA copolymer).

The comonomers present in the PHA copolymer are medium chain length hydroxyalkanoates and each of these, independently of one another, contains 6 or more carbon atoms. In one embodiment these may each independently contain from 6 to 16 carbon atoms, preferably from 6 to 14 carbon atoms, more preferably from 8 to 12 carbon atoms, e.g. 8, 10 or 12 carbon atoms.

The position of the hydroxy group within each monomer unit in the PHA copolymer may vary independently of one another, but typically these will be present at C-3, C-4, C-5 or C-6. In one embodiment the first, second and, where present, third comonomers can be independently selected from any medium chain length 3-hydroxy and 4-hydroxyalkanoates. The PHA copolymer may, for example, comprise both 3-hydroxyalkanoate and 4-hydroxyalkanoate units although, more typically, it will consist of either 3-hydroxyalkanoate or 4-hydroxyalkanoate units. Typically, each monomer unit will contain from 6 to 16 carbon atoms, preferably from 6 to 14 carbon atoms, more preferably from 8 to 12 carbon atoms, e.g. 8, 10 or 12 carbon atoms.

In one embodiment, the first, second and, where present, third comonomer units in the PHA copolymer will be a medium chain length 3-hydroxyalkanoate (3HA). These may contain from 6 to 16 carbon atoms, preferably from 6 to 14 carbon atoms, more preferably from 8 to 12 carbon atoms, e.g. 8, 10 or 12 carbon atoms. Suitable examples of 3HAs which may be present include those selected from the group consisting of 3-hydroxyoctanoate (3HO), 3-hydroxydecanoate (3HD) and 3-hydroxydodecanoate (3HDD).

In one embodiment the PHA copolymer is poly(3-hydroxyoctanoate-co-3-hydroxydecanoate), also referred to herein as “P(3HO-3HD)” or “P(3HO-co-3HD)”. This has the following structure:

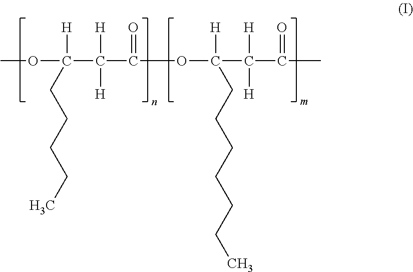

wherein n and m represent the number of repeating units of the 3HO and 3HD comonomers, respectively. In formula (I), n may range from about 300 to about 600, preferably from about 400 to about 500, e.g. about 450. In one embodiment, n may be 470. m may range from about 1,000 to about 1,300, preferably from about 1,100 to about 1,200, e.g. about 1,150. In one embodiment, m may be about 1,165. In one embodiment of formula (I), n=470 and m=1,165.

In one embodiment the PHA copolymer is poly(3-hydroxyoctanoate-co-3-hydroxydecanoate-co-3-hydroxydodecanoate), also referred to herein as “P(3HO-3HD-3HDD)” or “P(3HO-co-3HD-co-3HDD)”. This has the following structure:

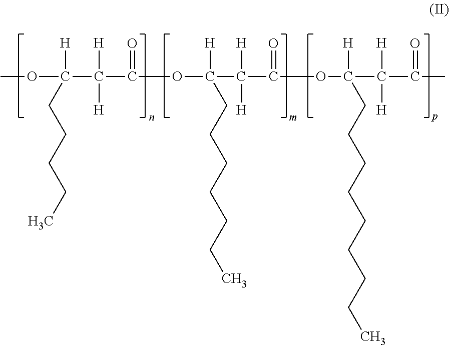

wherein n, m and p represent the number of repeating units of the 3HO, 3HD and 3-HDD comonomers, respectively. In formula (II), n may range from about 450 to about 800, preferably from about 550 to about 700, e.g. about 650. In one embodiment, n may be 630. m may range from about 700 to about 1,000, preferably from about 800 to about 900, e.g. about 850. In one embodiment, m may be about 860. p may range from about 200 to about 500, preferably from about 250 to about 400, e.g. about 350. In one embodiment, p may be 325. In one embodiment of formula (II), n=630, m=860, and p=325.

The chirality of the hydroxy-substituted carbon atom in each HA unit will be determined by the stereospecificity of the PHA biosynthetic enzymes used in the preparation of the polymer materials and will generally be of the R-configuration. Accordingly, the first blend component will preferably consist of R-hydroxyalkanoic acid monomers, e.g. R-3-hydroxyalkanoic acid monomers.

The PHA copolymer may include random or alternating repeating monomer units dependent on the method used for its production. Typically, however, it will be a random copolymer.

The molar ratio of comonomer units in the PHA copolymer may vary and will be dependent on the method used to produce the polymer. For example, the molar ratio will be determined by factors such as the type of microorganism and the biosynthetic pathway which it adopts to produce the copolymer, the nature of the feedstock used in its production, for example the carbon source and its concentration, the carbon source feeding strategy, the carbon to nitrogen ratio employed during biosynthesis, and other culturing process parameters.

In one embodiment, the PHA copolymer will contain 3-hydroxydecanoate (3-HD) monomer units and these will be present as the major component of the polymer. For example, where the PHA copolymer is a binary copolymer, the 3-hydroxydecanoate monomer units may be present in an amount ranging from 60 mol % to 85 mol %, preferably from 65 to 85 mol %, more preferably from 70 to 80 mol %, e.g. from 72 to 76 mol % (based on the weight average molecular weight, Mw, of the PHA copolymer). Where the PHA copolymer is a ternary copolymer, the 3-hydroxydecanoate monomer units may, for example, be present in an amount ranging from 40 mol % to 60 mol %, preferably from 40 to 55 mol %, more preferably from 45 to 55 mol %, e.g. from 47 to 50 mol % (based on the weight average molecular weight, Mw, of the PHA copolymer). High amounts of 3-hydroxydecanoate (3-HD) may be produced during the biosynthesis of MCL-PHAs by appropriate selection of the microorganism. For example, Pseudomonas species, such as Pseudomonas mendocina CH50, Pseudomonas putida LS46 and Pseudomonas putida KT2440, may be used to produce 3-hydroxydecanoate in high amounts in a PHA copolymer as herein described.

In one embodiment, the PHA copolymer will contain 3-hydroxyoctanoate (3-HO) monomer units. This may be present in an amount ranging from 15 to 40 mol %, preferably from 20 to 35 mol %, more preferably from 25 to 35 mol % (based on the weight average molecular weight, Mw, of the PHA copolymer). Where the PHA copolymer is a binary polymer, the 3-hydroxyoctanoate monomer units may be present in an amount from 20 to 35 mol %, preferably 20 to 30 mol %, e.g. from 24 to 28 mol % (based on the weight average molecular weight, Mw, of the PHA copolymer). Where the PHA copolymer is a ternary polymer, the 3-hydroxyoctanoate monomer units may be present in an amount from 20 to 40 mol %, preferably 25 to 35 mol %, e.g. from 30 to 35 mol % (based on the weight average molecular weight, Mw, of the PHA copolymer).

In one embodiment, the PHA copolymer will contain 3-hydroxydodecanoate (3-HDD) monomer units. This may be present in an amount ranging from 10 to 30 mol %, preferably from 15 to 25 mol %, e.g. 17 to 22 mol % (based on the weight average molecular weight, Mw, of the PHA copolymer). Where such monomer units are present, the PHA copolymer will generally be a ternary polymer.

Molecular weight ranges for the MCL-PHA copolymers will be dependent on the method used in their production and may be adjusted accordingly. For example, the molecular weight will be determined by factors such as the type of microorganism and the biosynthetic pathway which it adopts to produce the PHA copolymer, the nature of the carbon source, process parameters, etc. Suitable molecular weights may range from 50 to 600 kDa, preferably from 150 to 500 kDa, more preferably from 200 to 400 kDa, e.g. from 200 to 350 kDa or from 300 to 350 kDa. In one embodiment, the molecular weight of the PHA copolymer may be about 330 to about 340 kDa.

The second component of the polymer blends is either a PHA homopolymer containing short chain length hydroxyalkanoate monomer units, or a polylactide (PLA).

In one embodiment the second component of the blend is a PHA homopolymer containing short chain length hydroxyalkanoate monomer units. The short chain length PHA may have 3, 4 or 5 carbon atoms in the repeating unit. Preferably this will have 3 carbon atoms in the repeating unit.

In one embodiment the PHA homopolymer is poly(3-hydroxybutyrate) or “P(3HB)” which is a homopolymer of 3-hydroxybutyric acid units. P(3HB) has the following structure:

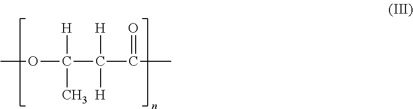

wherein n represents the number of repeating units of the 3HB monomer. In formula (III), n may range from about 3,000 to 23,000, preferably from about 3,400 to about 22,700, e.g. about 5,800 to about 11,600.

Molecular weight ranges for the SCL-PHA polymer will be dependent on the method used in their production and may be adjusted accordingly. For example, the molecular weight will be determined by factors such as the type of microorganism and the biosynthetic pathway which it adopts to produce the PHA homopolymer, the nature of the carbon source, process parameters, etc. Suitable molecular weights may range from 200 kDa to 2 MDa, preferably from 300 kDa to 2 MDa, e.g. from 350 kDa to 1 MDa, from 500 kDa to 1 MDa, or from 350 kDa to 500 kDa.

In another embodiment the second component may be a polylactide comprising lactic acid repeating units. Poly(lactic acid) or “PLA” is a biodegradable semi-crystalline polyester which may exist in different stereoisomeric forms: L- and D-lactic acid. For use in the invention, the PLA may take any suitable form selected from poly(L-lactic acid) (“PLLA”), poly(D-lactic acid) (“PDLA”), and racemic products, i.e. poly(D,L-lactide). Preferred for use in the invention is PLLA.

PLA polymers are available commercially from suppliers such as CORBION, GoodFellow, and Sigma. Those which are commercially available may have molecular weights as follows: PLLA—100 kDa to 260 kDa; PLDA—about 124 kDa; PDLA 10 to 28 kDa. In one embodiment the PLA polymer is PL38 PURASORB which is a homopolymer of L-lactic acid supplied by Corbion (Netherlands).

The PHA blends for use in the invention comprise: (a) from 5 to 40 wt. % of a first component which is a PHA copolymer comprising two or more different medium chain length hydroxyalkanoate monomer units; and (b) from 60 to 95 wt. % of a second component which is either a PHA homopolymer containing a short chain length hydroxyalkanoate monomer unit, or a polylactide (PLA). Any combination of any of the first and second blend components herein described may provide a PHA blend for use in the invention. Such PHA blends are in themselves novel and any of the blends herein described form a further aspect of the invention.

In one embodiment the PHA blend may comprise a first component which is a PHA copolymer comprising two or more different medium chain length HAs, and a second component which is poly(3-hydroxybutyrate) (or “P(3HB)”).

In another embodiment the PHA blend may comprise a first component which is selected from poly(3-hydroxyoctanoate-co-3-hydroxydecanoate) (or “P(3HO-3HD)”) and poly(3-hydroxyoctanoate-co-3-hydroxydecanoate-co-3-hydroxydodecanoate) (or “P(3HO-3HD-3HDD)”), and a second component which is poly(3-hydroxybutyrate) (or “P(3HB)”).

In one embodiment, the PHA blend may comprise a first component which is a PHA copolymer comprising two or more different medium chain length HAs, and a second component which is a polylactide.

In another embodiment the PHA blend may comprise a first component which is selected from poly(3-hydroxyoctanoate-co-3-hydroxydecanoate) (or “P(3HO-3HD)”) and poly(3-hydroxyoctanoate-co-3-hydroxydecanoate-co-3-hydroxydodecanoate) (or “P(3HO-3HD-3HDD)”), and a second component which is a polylactide.

Non-limiting examples of suitable PHA blends include the following:
P(3HO-3HD)/P(3HB)
P(3HO-3HD-3HDD)/P(3HB)
P(3HO-3HD-3HDD)/PLA
P(3HO-3HD)/PLA
P(3HO-3HD-3HDD)/PLLA
P(3HO-3HD)/PLLA
P(3HO-3HD-3HDD)/PDLA
P(3HO-3HD)/PDLA

The first component forms the minor component of the blend and is present in an amount ranging from 5 to 40 wt. % (based on the total weight of the blend). More specifically, this may be present in an amount ranging from 10 to 35 wt. %, preferably from 15 to 30 wt. %, more preferably from 18 to 32 wt. %, e.g. from 20 to 30 wt. %. Where the second component is a PHA homopolymer, the amount of the first component may range from 15 to 35 wt. %, preferably from 18 to 32 wt. %, e.g. from about 20 to about 30 wt. %. Where the second component of the blend is a polylactide, the amount of the first component may range from 5 to 20 wt. %, preferably from 10 to 18 wt. %, more preferably from 12 to 18 wt. %, e.g. about 15 wt. %.

The second component forms the major component of the blend and is present in an amount ranging from 60 to 95 wt. % (based on the total weight of the blend). More specifically, this may be present in an amount in the range from 65 to 90 wt. %, preferably from 65 to 85 wt. %, more preferably from 68 to 82 wt. %, e.g. from about 70 to about 80 wt. %. Where the second component of the blend is a PHA homopolymer, this may be present in an amount in the range from 60 to 90 wt. %, preferably 65 to 85 wt. %, e.g. about 70 to about 80 wt. %. Where the second component of the blend is a polylactide, this may be present in an amount in the range from 75 to 95 wt. %, preferably 80 to 90 wt. %, e.g. about 85 wt. %.

In one embodiment of the invention the PHA blends are binary blends containing only the first and second polymer components herein defined. In other embodiments, however, these may contain other known components such as other polymers, fillers or additives as desired. Other biodegradable polymers which may be present include polysaccharides such as cellulose and its derivatives (e.g. hydroxypropylcellulose, methylcellulose, cellulose acetate, etc.), starch and its derivatives, chitosan, alginate, hyaluronic acid, pectin, carrageenans, agarose, and chondroitin sulphate; proteins such as collagen, gelatine, elastin, albumin, fibrin, and natural polyamino acids (e.g. poly-glutamic acid, poly-lysine, etc.); synthetic polyesters such as polydioxanone, poly(trimethylene carbonate); polyurethanes; poly(ester amide)s; polyanhydrides; poly(anhydride-co-imide); polyphosphazenes; and polyphosphoesters. Fillers or additives which may be present include plasticizers (e.g. fatty acids, soybean oil, sorbitol, PEG, oleic acid, citric acid, tartaric acid, malic acid, etc.), and stiffening or reinforcing agents such as synthetic and natural clay minerals, graphene and its derivatives, carbon nanotubes, silica, hexagonal and tubular boron nitride, double layered hydroxides, polyhedral oligomeric silsesquioxane (POSS), monocrystalline and nanofibrillated cellulose, starch, chitosan, bioactive glass, and phosphate glass. Fillers can be introduced as reinforcing additives to change the mechanical properties and/or as modifiers of the kinetics for the release of any active agents.

Other additives may include appropriate visualisation materials, for example radiopaque materials such as barium sulphate, or therapeutic agents which may be selected from immunosuppressive, anti-inflammatory, anti-thrombogenic, anti-proliferative agents, anti-cancer drugs and antibiotics. Examples of suitable therapeutic agents include, but are not limited to, antibodies, rapamycin, everolimus, zotarolimus, tacrolimus, aspirin, dexamethasone, and paclitaxel.

PHAs suitable for use in the invention and methods for their production are generally known in the art. The PHAs can be prepared from a biological source such as a microorganism which naturally produces PHAs and which can be induced to produce the desired PHAs by adjusting the culture conditions and feedstocks. PHAs may be produced in natural or genetically engineered microorganisms.

PHAs are derived from microorganisms, typically from bacteria, by culturing in a bioreactor under conditions in which the supply of nutrients is limited. In such methods, a culture of the microorganism in a suitable medium is fed appropriate nutrients so that it multiplies rapidly. Once the microorganism has multiplied to a sufficient level, the nature of the nutrient composition is changed to force the microorganism to synthesise PHA. Biosynthesis of PHAs may be induced by limiting the supply of nutrients such as phosphorus, nitrogen, and trace elements, by reducing the supply of oxygen, or by an excess supply of carbon sources. The PHAs are deposited in the form of highly refractive granules in the cells and can be recovered by disruption of the cells.

The choice of different carbon sources, feeding strategies, media compositions and bacterial strains will ultimately define the final type of polymer and can be adjusted accordingly. Metabolic preferences of any given strain towards carbon sources and thus the production of certain PHAs are generally known in the art or can readily be determined by those skilled in the art. For example, when using Pseudomonas sp, the use of a structurally related carbon source, such as octanoic acid, typically results in the production of either a P(3HO) homopolymer or a P(3HO-3HD) copolymer, with 3HO present in the greater amount. On the other hand, the use of an unrelated carbon source (e.g. glucose) forces the microorganism to undergo a different metabolic pathway and produce different MCL-PHA monomer units. For any given bacterial strain, adjustment of the media composition, feeding strategies and/or growth conditions can be used to tailor the composition of the PHA product including the ratio of monomers in any resulting copolymer.

Any strain of bacteria known in the art that produces PHAs can be used for producing the PHAs for use in the invention. Various microorganisms are known for use in the production of PHAs. This includes both Gram-negative and Gram-positive bacteria, although Gram-negative bacteria are mainly used. Those generally used for the production of short-chain length PHAs (SCL-PHAs) include Cupriavidus necator (formerly known as Ralstonia eutropha), Alcaligenes latus, Bacillus cereus, Aeromonas caviae, Rhodospirillum rubrum, Methylobacterium extorquens, Halomonas boliviensis LC 1, Bacillus subtilis, and Bacillus megaterium. Those typically used for the production of medium-chain length PHAs (MCL-PHAs) and their copolymers include those of Pseudomonas genus, for example Pseudomonas putida, Pseudomonas oleovorans, Pseudomonas mendocina CH50, Pseudomonas fluorescence, Pseudomonas aeruginosa, Pseudomonas raguenesii, Pseudomonas guezennei, Pseudomonas stutzeri, and Pseudomonas cepacia. Comamonas species, such as Comamonas testosteronii, may also be used.

Suitable microorganisms for use in the invention are commercially available from various sources, such as NCMB or ATCC.

In the production of SCL-PHAs, for example P(3HB), the use of the Gram-positive bacterium Bacillus subtilis, e.g. Bacillus subtilis OK2, has been found to be particularly beneficial. Bacillus subtilis OK2 is commercially available from various sources, e.g. from the National Institute of Genetics. It has also been deposited by the Applicant at the National Collection of Industrial and Marine Bacteria (NCIMB), which serves as an International Depositary Authority (IDA), on 1 Sep. 2017, under accession number NCMB 42804. Bacillus subtilis is classified as GRAS. As a Gram-positive bacterium it also lacks lipopolysaccharides (LPS) and thus the polymer produced will lack LPS, which is a strong immunogen, and hence be inherently non-immunogenic. It has been found to provide SCL-PHAs in good yield with a high molecular weight. Its use in the production of the PHA homopolymer (e.g. P(3HB)) represents a preferred embodiment of the invention.

Pseudomonas mendocina CH50 has been found to be particularly suitable for use in the invention for the production of MCL-PHAs, at least in part due to its versatility with respect to the choice of carbon source. Specifically, the inventors have found it has the ability to use a range of carbon sources for the production of PHAs other than glucose. Suitable carbon sources may include vegetable oils, carbohydrates, and fatty acids, as well as sugarcane molasses and biodiesel waste. The use of Pseudomonas mendocina CH50 to produce PHA copolymers as herein described using a carbon source other than glucose, and the resulting PHA copolymers form further aspects of the invention.

In another aspect, the invention thus provides a method of producing a PHA copolymer, preferably a PHA copolymer comprising two or more different medium chain length HAs (e.g. P(3HO-3HD) or P(3HO-3HD-3HDD)), said method comprising the steps of: culturing Pseudomonas mendocina CH50 in a culture medium which includes a carbon source other than glucose; harvesting biomass from the culture medium; extracting PHA from the harvested biomass; and optionally purifying the crude PHA whereby to obtain a purified PHA.

In yet another aspect the invention provides a PHA copolymer, for example P(3HO-3HD) or P(3HO-3HD-3HDD), obtained or obtainable by culturing Pseudomonas mendocina CH50 in the presence of a culture medium which includes a carbon source other than glucose. The PHA copolymer may be a copolymer as herein defined in respect of any of the embodiments relating to the PHA blend.

Microorganisms may be cultured to produce PHAs using any conventional methods for bacterial cultivation, including batch-mode and continuous mode bioreactor cultivation. During culturing of the microorganisms the conditions are carefully controlled. This includes control over appropriate levels of nutrients, dissolved oxygen, temperature and pH. Required nutrients include carbon, nitrogen, and phosphorus as well as mineral salts. Suitable conditions for culturing may readily be determined by those skilled in the art taking into account considerations such as the nature of the microorganism and its optimum growth conditions. Typically, the pH will be in the range of from 6.0 to 7.0, preferably from 6.5 to 7.0, e.g. about 7.0. The temperature for culturing will typically range from about 30 to 35° C., for example it may be about 30° C. Mixing speeds and time of mixing should be sufficient to allow the microorganism to proliferate and to allow PHA to be synthesised. Suitable mixing speeds may be in the range from 150 to 200 rpm, e.g. about 200 rpm, and mixing times may range from about 12 to about 48 hours, e.g. about 48 hours.

The choice of nutrient media will be dependent, at least in part, on the choice of microorganism—this depends on the metabolic pathway used by the organism for PHA synthesis —but it will include both carbon and nitrogen sources, as well as salts and minerals and other trace elements.

The use of different carbon sources will define different monomer unit compositions within the polymer chain and thus the properties of the obtained PHA materials. Suitable carbon sources include any of the following, and any mixtures thereof:
Carbohydrates such as glucose, sucrose, fructose, galactose, arabinose and xylose, or mixtures thereof,
Lipids such as those derived from vegetable oils, coconut oil, walnut oil, corn oil, rapeseed oil, hazelnut oil, olive oil, groundnut oil, fish oil, etc. Examples of suitable lipids include C₆₋₁₈ fatty acids, preferably C₂₋₁₈ fatty acids, and their salts, e.g. hexanoic acid, heptanoic acid, octanoic acid, nonanoic acid, decanoic acid, undecanoic acid, dodecanoic acid, and their salts;
Alcohols such as ethanol, glycerol, n-propanol, n-butanol, 1,4-butane-diol, propylene glycol;
Organic acids such as acetate, propionate, butyrate, valerate, lactate, citrate, etc.
Amino acids, such as glutamate, histidine, asparagine, etc.
Waste materials such as by-products of biodiesel production (e.g. crude glycerol);
Complex carbohydrates, e.g. sugarcane molasses;
Yeast extract;
Other organic molecules such as γ-butyrolactone.

Mixtures of carbohydrates and fatty acids are generally preferred for use in production of the PHA polymer materials.

A preferred carbon source for use in the production of both SCL and MCL-PHAs is glucose. For the production of MCL-PHAs, for example using the bacterial strain Pseudomonas mendocina CH50, coconut oil is a preferred carbon source.

Suitable nitrogen sources include ammonium salts such as ammonium sulphate, ammonium chloride, and ammonium hydroxide, urea, and yeast extract.

PHA production generally involves a two-stage procedure—a first stage involving rapid cell growth followed by a second accumulation stage. The nature of the nutrient media for use in the different stages of the procedure will differ. Exhaustion of one or more types of nutrients in the second stage results in the accumulation of PHAs. The use of nitrogen-limiting conditions may be used to force the organism to use the carbon source and synthesise PHAs.

For the production of SCL-PHAs, a modified Kannan and Rehacek media (K-R) may be used (see Kannan LV and Rehacek Z, Indian J. Biochem. 7: 126-129, 1970). This has the following composition (g/L): ammonium sulphate 5.0, potassium chloride 3.0, yeast extract 2.5, glucose 35.0.

A suitable production medium for use in the production of MCL-PHAs is a Mineral Salt Medium (MSM). This may be used in a two-stage culture as follows:
MSM media composition (first stage) (g/L): ammonium sulphate 0.45, potassium phosphate monobasic 2.38, sodium hydrogen phosphate 3.42, magnesium sulphate 0.4, carbon source 20.0, trace elements 1 mL/L.
MSM media composition (production stage) g/L: ammonium sulphate 0.50; potassium phosphate monobasic 2.65; sodium hydrogen phosphate 3.80; magnesium sulphate 0.4, carbon source 20.0, trace elements 1 mL/L.

An example of a trace element solution for use in the culture medium is (amounts provided in g/L): cobalt (II) chloride 0.22; iron (III) chloride 9.70; calcium chloride 7.80; nickel(III) chloride 0.12; chromium (VI) chloride 0.11; copper sulphate pentahydrate 0.16.

The monomeric composition and the molecular weight of the PHA materials can be controlled by complying with an established culturing protocol.

PHA is produced by disrupting the cells followed by extraction of the PHA from the disrupted cells. Standard methods known in the art can be used to disrupt the cells and release the PHAs produced, for example this may be done mechanically (e.g. by homogenisation), or chemically (e.g. by the addition of sodium hypochlorite). Collection of the biomass may be carried out using methods such as centrifugation which results in the formation of cell pellet. The harvested biomass is then washed, e.g. with water and frozen under standard conditions, e.g. at −20° C. before lyophilisation. For extraction and purification of PHA methods known in the art can be used. For example, the lyophilised cell mass may be mixed with an organic solvent such as chloroform, methylene chloride, or pyridine in order to extract the PHA using a soxhlet based extraction carried out at the boiling point of the solvent. Chloroform is generally preferred (boiling point: 65° C.). The polymer solution may be concentrated by standard methods such as rotary evaporation. The polymer can be precipitated from this solution using standard methods, for example the PHA solution may be added to cold organic solvent, such as methanol, ethanol, acetone, ether or hexane. The use of cold methanol is generally preferred. The polymer can be collected and, if desired, washed with additional solvent, e.g. methanol.

Extraction of PHA via the soxhlet method may, for example, be carried out as follows: after 48 hours of incubation, the cells are harvested by centrifugation at 4600 rpm for 30 minutes. The cell pellet is lyophilised prior to the extraction. Dried biomass is placed in a thimble within the soxhlet apparatus and refluxed for 24 hours with methanol at 70° C. This step is carried out to remove all the impurities soluble in methanol. Methanol is then replaced with chloroform, and the cells are refluxed for another 24 hours to facilitate PHA dissolution in the chloroform solution at 65° C. The chloroform solution is concentrated using a rotary evaporator. PHA is precipitated out using an ice cold solution of methanol.

Suitable lactic acid polymers for use in the invention are generally known in the art and are commercially available. These include PL38 which is a medical grade PLLA available from Corbion.

The PHA blends herein described are immiscible blends. These may be prepared using any of the conventional processes used in the plastics industry, including but not limited to, any of the following: melt blending using extrusion or blending in solutions. Blending in solution is generally preferred in order to retain the property of the materials. Processing temperatures will be dependent on the thermal properties of the blend components and can be selected accordingly. If using melt blending techniques, care should be taken to optimise the process to minimise the exposure of the materials to elevated temperatures since this can lead to deterioration of the materials' properties. The temperature for melt blending is defined by the melt temperature of the component in the blend having the highest melting temperature. In the blends herein described, the P(3HB) and PLA (e.g. PLLA) will typically have the highest melting temperatures (Tm about 175° C.). Suitable temperatures for melt blending may be in the range from 190 to 200° C.

Blending in solution may be carried out by adding the desired quantities of blend components to an organic solvent, such as chloroform, and stirring the solution to dissolve the polymer materials. Temperature and duration of stirring will depend on the nature of the blend components (e.g. their viscosity) and may readily be determined. For example, the temperature may range from 20 to 100° C., e.g. from 20 to 50° C. In one embodiment, blending may be carried out at ambient temperature. Stirring may be carried out for about 10 to 60 minutes, or longer (e.g. overnight). The duration of stirring will depend on the temperature and concentration of the components of the blend and may be shortened by blending at a higher temperature. Evaporation of the solvent results in precipitation of a PHA polymer blend. This may be filtered and dried, e.g. under vacuum conditions at elevated temperature.

As will be understood, the stent body or scaffold will be generally cylindrical and balloon-expandable, i.e. capable of radial expansion in a body lumen. It may be any stent design. For example, it may be composed of a scaffold that comprises a pattern or network of interconnecting structural elements (or “struts”) formed from a polymeric tube of polymeric material. The stent precise design is not critical to the invention.

As described herein, the stent body may be provided with a coating that includes a therapeutic agent.

The blends herein described may be processed into a stent or component of a stent (e.g. a stent body or scaffold) using known manufacturing processes. Processes for producing a generally cylindrical stent body include injection moulding, extrusion, dip moulding using polymer solutions, film casting from polymer solutions, electrospinning, compression moulding, photochemical etching, lithography, and fused deposition modelling. Techniques such as extrusion and dip moulding from polymer solutions are particularly suitable for the production of hollow, thin-walled structures. 3D printing techniques may also be used.

Formation of a stent scaffold from a tube may be achieved using known techniques such as laser ablation of a stent pattern in the tube. Any stent design may be used. The stent scaffold may then be crimped onto a delivery balloon and sterilised using conventional methods such as gamma-radiation prior to final packaging ready for use.

In one embodiment, the stents may carry one or more active drug substances capable of release following delivery of the stent to the target site. Active agents may be incorporated into the PHA blend during production of the blend or, more typically, these may be provided in the form of a coating over the stent scaffold prior to crimping of the stent scaffold onto a delivery balloon. Methods for the application of a drug-containing coating to a stent scaffold are known in the art and include methods such as mixing of the drug substance with a solvent and a polymer dissolved in the solvent to produce a solution which is applied to the stent scaffold (for example by spraying, dipping, or immersing) followed by evaporation of the solvent. Suitable solvents include chloroform, methylene chloride and acetone. Evaporation of the solvent leaves a drug-eluting polymeric coating on the surface of the stent. The inclusion of a polymer in the coating allows the drug to be retained on the stent during expansion and may also delay delivery of the drug in vivo. Suitable polymeric coatings are known in the art and include appropriate bioabsorbable polymers such as poly(L-lactic acid), poly(lactic acid-co-glycolic acid), polycaprolactone, PHAs such as poly(hydroxybutyrate), poly(hydroxybutyrate-co-hydroxy-valerate).

Drugs which may be incorporated into the stents of the invention include, but are not limited to, any of the following: antiplatelet agents, anticoagulants, antimitotic agents, antioxidants, antimetabolites, anti-inflammatory agents, immunosuppressive, anti-inflammatory, anti-thrombogenic, anti-proliferative agents and antibodies. Examples of such drugs include rapamycin, everolimus, zotarolimus, tacrolimus, aspirin, dexamethasone, and paclitaxcel. Suitable antibodies include, but are not limited to, anti-CD34 antibodies and anti-platelet GPIIb/IIIa antibody.

In use, the stent of the invention is introduced transluminally to the selected target body lumen and radially expanded into contact with the walls of the lumen. The lumen may be a cavity of any tubular organ although typically it will be a blood vessel. Transluminal delivery will typically be achieved using a catheter specially designed for the delivery of stents and radial expansion will generally be achieved by balloon expansion.

The PHA blends herein described have favourable properties for use in the production of stents. At least in some embodiments, such properties include one or more of the following:
High biocompatibility compared to established synthetic polymers.
The PHAs degrade by surface degradation and hence lead to long term stability of the implant compared to bulk degrading materials such as PLLA/PGA where the structure begins to loose stability due to bulk degradation.
The degradation products of PHAs are hydroxyalkanoic acids which are much less acidic than lactic acid/glycolic acid which are produced on degradation of PLLA/PGA implants and which cause inflammation.
The degradation products of PHAs are known metabolites already present in the body and hence are completely non-immunogenic in nature.
The degradation rate and mechanical properties of the PHA blends can be tailored to suit the patient condition.

The mechanical properties of the materials provide the desired scaffold properties such as radial strength and elasticity. Mechanical properties include tensile strength which is a measure of the capacity of the material to withstand a loading tending to elongate it, Young's modulus which is a measure of the stiffness of a material, and elongation at break which is a measure of the elastomeric properties of a material.

In at least some embodiments, the tensile strength (a) of the PHA blends herein described, as measured according to the method described in the examples, is greater than 20 MPa, preferably in the range of from 30 to 50 MPa.

In at least some embodiments, the Young's modulus (E) of the PHA blends herein described, as measured according to the method described in the examples, is greater than 1 GPa.

In at least some embodiments, the elongation at break (sb) of the PHA blends herein described, as measured according to the method described in the examples, is at least 10%. Elongation at break is particularly important due to the need for the stent to expand in situ.

Due to the limited compatibility of the components of the blends, their thermal properties will be similar to that of the components. On the other hand, mechanical properties are strongly dependent on the nature of the blend composition and can be tailored according to need, for example, by adjusting the ratio of first and second components, adjusting Mw/Mn of individual components, and adjusting the mol % of monomers in the PHA copolymer. The blends become more pliable, less stiff and weaker with an increase in the content of the first component.

### EXAMPLES

The invention will now be described in more detail by way of the following non-limiting examples and with reference to the accompanying figures, in which:

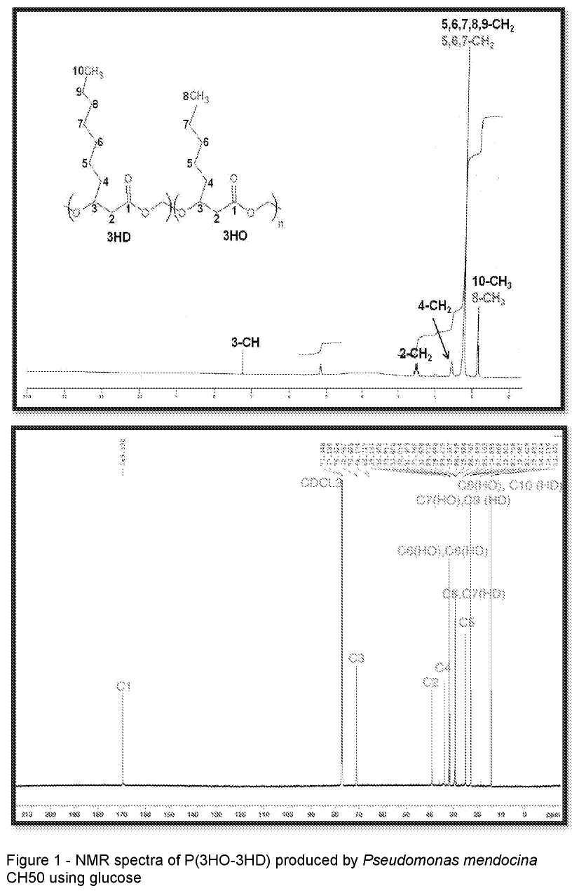

FIG. 1 shows the ¹H NMR (upper panel) and ¹³C NMR (lower panel) of the P(3HO-3HD) polymer produced by P. mendocina CH50 with glucose as the carbon source in Example 2.

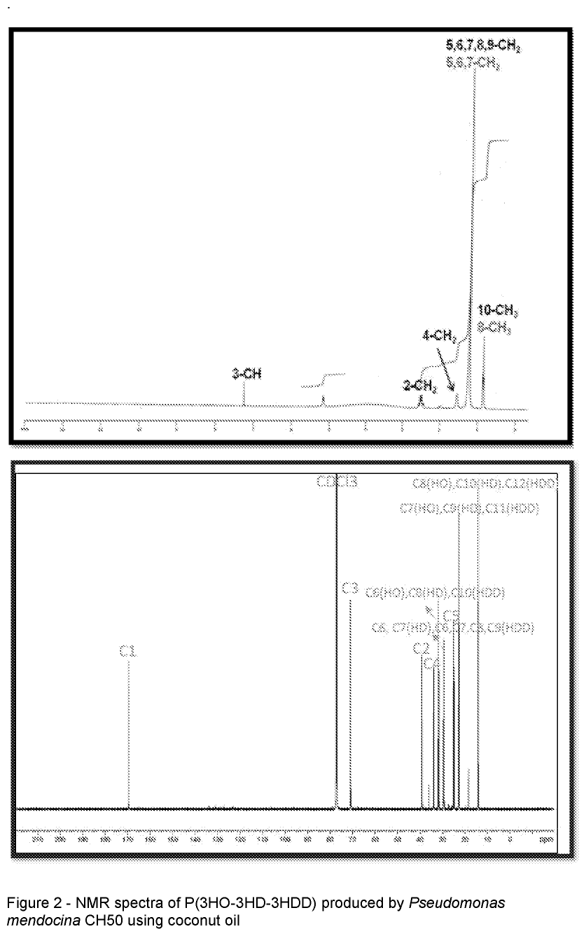

FIG. 2 shows the ¹H NMR (upper panel) and ¹³C NMR of the P(3HO-3HD-3HDD) polymer produced by P. mendocina CH50 with coconut oil as the carbon source in Example 3.

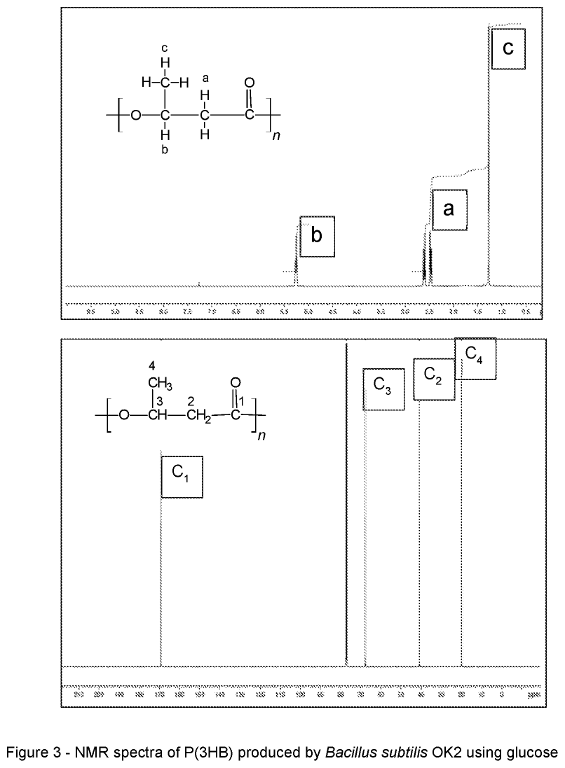

FIG. 3 shows the ¹H NMR (upper panel) and ¹³C NMR (lower panel) of the P(3HB) polymer produced by Cupriavidus necator with walnut oil as carbon source in Example 8.

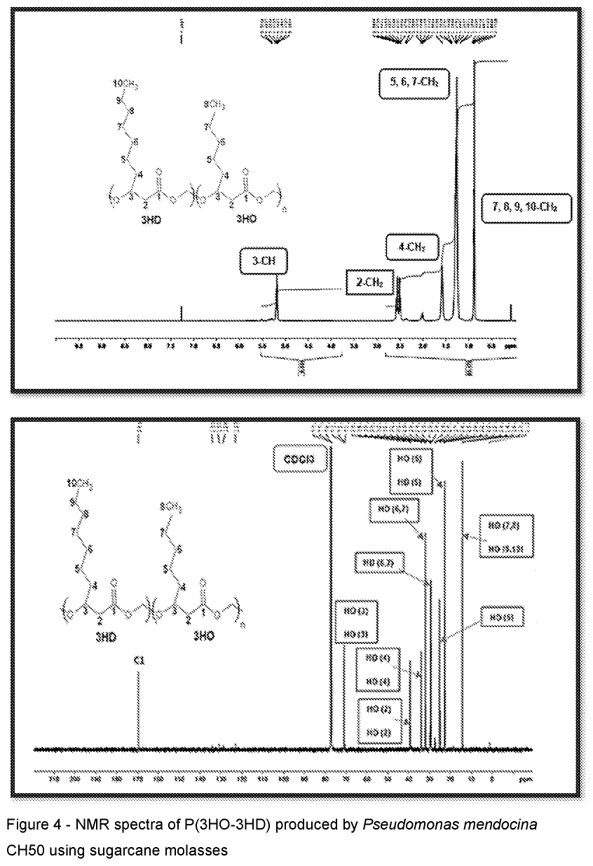

FIG. 4 shows the ¹H NMR (upper panel) and ¹³C NMR (lower panel) of the P(3HO-3HD) polymer produced by Pseudomonas mendocina CH50 with sugarcane molasses as carbon source in Example 9.

### Measurement of Mechanical Properties:

In the following examples, tensile strength, Young's modulus and elongation at break are determined using a 5942 Testing Systems (Instron) equipped with 500 N load cell at room temperature. The test is conducted using films which are 5 mm in width and 3.5 to 5.0 cm in length. Before measurement, the thickness and width of the specimen are measured in several places and an average value used to calculate the cross-sectional area. The gauge length of the sample holder is set at 23 mm and a deformation rate of 5 mm per minute for SCL-PHA based materials and 10 mm per minute for MCL-PHA based materials is employed. Young's modulus, tensile strength and elongation at break are calculated from the stress-strain curve and average values calculated for 3-6 specimens. Data analysis is carried out using BlueHill 3 software.

### Measurement of Molecular Weight:

Unless otherwise specified, the molecular weight of the polymer materials is determined using a PLgel 5 m MIXED-C (300×7.5 mm) column which is calibrated using narrow molecular weight polystyrene standards from 162 Da to 15,000 kDa. The eluent used was chloroform. 5 mg/mL of the polymer is introduced into the GPC system at a flow rate of 1 mL/min. The eluted polymer is detected with a refractive index detector. Data is collected and analysed using “Agilent GPC/SEC” software.

### Example 1—Production and Characterisation of Poly(3-Hydroxybutyrate) “P(3HB)” from Glucose by Bacillus subtilis OK2

Producer organism: Bacillus subtilis OK2 (obtained from the National Institute of Genetics).

Media Composition:

Production Media
Ammonium sulphate: 5 g/L
Potassium chloride: 3 g/L
Yeast extract: 2.5 g/L (autoclaved at 121° C. for 15 minutes)

Carbon Source
Glucose: 35 g/L (autoclaved at 110° C. for 11 minutes)
pH of all the media components was adjusted to 6.8

Production: A single colony of Bacillus subtilis OK2 was used to inoculate the autoclaved nutrient broth. This nutrient broth was incubated for 16 hours at 30° C. at 150 rpm. Glucose was used as the sole carbon source. Autoclaved production media (modified Kannan and Rehacek media) was inoculated using the nutrient broth as the seed culture. Inoculated production media was then incubated for 48 hours at 30° C. at 200 rpm.

Harvesting: The cells were harvested at 48 hours by centrifugation at 4600 rpm for 30 minutes. They were washed thrice, first with distilled water followed by 10% ethanol and then again with distilled water. The cells were homogenized using a homogenizer for approximately 15 minutes. The cells were then kept at −20° C. overnight after which they were placed in a freeze dryer for lyophilisation.

Extraction: Polymer was extracted from the cells using the soxhlet extraction method. The cells were treated in a soxhlet apparatus with methanol for 24 hours, under reflux conditions, as a washing step to remove the impurities. After this, the methanol was replaced with chloroform which was used to extract the polymer from the cells. The cells were treated with chloroform for 4 hours under reflux conditions. This chloroform solution was concentrated using a rotary vacuum evaporator and the polymer was precipitated using ice-cold methanol solution. Extraction was continued using the same batch of cells by incubating them twice with chloroform for 24 hours under reflux conditions. This chloroform solution was again concentrated using the rotary vacuum evaporator and the polymer was precipitated using ice-cold methanol solution.

Mechanical properties: Film samples were prepared by casting 10 ml of 10 w/v % polymer solution in chloroform into a glass petri dish (6 cm diameter). The samples were left covered at room temperature until solvent evaporation was complete (monitored by weight change). Mechanical properties were determined using tensile testing with samples cut from the solvent-cast film into strips of 5 mm width and 40 mm length. The thickness of the sample was around 300 μm. The crosshead speed was 10 mm/min.

Elongation at break (ε_{U}) was determined as 24%. Young's modulus (E) of P(3HB) was determined as 1.3 GPa. Ultimate tensile strength (σ_{U}) reached 26 MPa.

### Example 2—Production and Characterisation of Poly(3-Hydroxyoctanoate-Co-3-Hydroxydecanoate) “P(3HO-3HD)” from Glucose by Pseudomonas mendocina CH50

Producer organism: Pseudomonas mendocina CH50 obtained from the National Collection of Industrial and Marine Bacteria, NCIMB (deposit No. 10542).

Media Composition:

Second Stage (Mineral Salt Medium)
Ammonium sulphate: 0.45 g/L
Sodium hydrogen phosphate: 3.42 g/L
Potassium dihydrogen phosphate: 2.38 g/L

Production Media (Mineral Salt Medium)
Ammonium sulphate: 0.50 g/L
Sodium hydrogen phosphate: 3.80 g/L
Potassium dihydrogen phosphate: 2.65 g/L

Carbon Source
glucose: 20 g/L
Trace element solution: 1 ml/L
Magnesium sulphate heptahydrate: 0.8 g/L

Production: A single colony of Pseudomonas mendocina CH50 was used to inoculate the autoclaved nutrient broth. This nutrient broth was incubated for 16 hours at 30° C. at 150 rpm. Autoclaved second stage media was inoculated using the nutrient broth culture as the seed culture. Inoculated second stage media was then incubated at 30° C. at 150 rpm until the optical density reached 1.6 without dilution. This was used as the inoculum to inoculate the production media (10% culture volume). Inoculated production media was then incubated for 48 hours at 30° C. at 200 rpm.

Harvesting: The cells were harvested at 48 hours by centrifugation at 4600 rpm for 30 minutes. They were washed thrice, first with distilled water followed by 10% ethanol and then again with distilled water. The cells were homogenized using a homogenizer for approximately 15 minutes. The cells were then kept at −20° C. overnight after which they were placed in a freeze dryer for lyophilisation.

Extraction: Polymer was extracted from the cells using the soxhlet extraction method. The cells were incubated in the soxhlet with methanol for 24 hours under reflux conditions as a washing step to remove the impurities. After this, the methanol was replaced with chloroform which was used to extract the polymer from the cells. Extraction was continued using the same batch of cells by incubating them with chloroform for 24 hours under reflux conditions. This chloroform solution was concentrated using a rotary vacuum evaporator and the polymer was precipitated using ice-cold methanol solution. Yield: 43%, dry cell weight 0.57 g/l.

Characterisation: The resulting polymer was characterised by GC-MS and NMR. The GC-MS results are presented in Table 1:

TABLE 1

| | HO content | HD content |
|---|---|---|
| | (mol %) | (mol %) |
| Mean | 26.2 | 73.8 |
| SD | 1.0 | 1.7 |

¹H NMR and ¹³C NMR are presented in Figure 1.

Thermal and morphological properties: The resulting P(3HO-3HD) polymer is a semi-crystalline polymer characterized by slow crystallization. Melting of the crystalline phase was observed only for aged samples. The crystalline phase was not detected by differential scanning calorimetry after melting of the polymer in a first heating cycle and cooling the sample at the rate of 20 K/min. The glass transition temperature determined by differential scanning calorimetry was in the range between −41° C. to −46° C. The crystalline phase of P(3HO-3HD) melted between 26 to 66° C. The highest melting rate (endothermic peak maximum) was 57±2° C. Enthalpy of fusion (crystallinity degree) changed with polymer storage and for polymer aged at room temperature (for a period of 5 weeks) reached 27±2 J/g.

TABLE 2

| PHA | Tg (° C.) | Tm (° C.) | ΔH (J/g) |
|---|---|---|---|
| P (3HD-co-3HO) | −45.1 | 54.3 | 19.0 |

Mechanical properties: Film samples were prepared by casting 10 ml of 10 w/v % polymer solution in chloroform into a glass petri dish (6 cm diameter). The samples were left covered at room temperature until solvent evaporation was complete (monitored by weight change). Mechanical properties were determined by tensile testing using samples cut from the solvent-cast film into strips of 5 mm width and 40 mm length. The thickness of the sample was around 300 m. The crosshead speed was 10 mm/min.

P(3HO-3HD) is a soft ductile polymer with elongation at break (ε_{U}) 580±30%. Young's modulus (E) of P(3HO-3HD) was determined as 8.7±1.1 MPa. Ultimate tensile strength (σ_{U}) reached 10.4±1.0 MPa.

Molecular weight (Mw) and polydispersity index (PDI) were determined by GPC. Mw was determined as 340 kDa and PDI as 2.7.

### Example 3—Production and Characterisation of Poly (3-Hydroxyoctanoate-Co-3-Hydroxydecanoate-Co-3-Hydroxydodecanoate) “P(3HO-3HD-3HDD)” from Coconut Oil by Pseudomonas mendocina CH50

Producer organism: Pseudomonas mendocina CH50 obtained from NCIMB.

Media Composition:

Second Stage (Mineral Salt Medium)
Ammonium sulphate: 0.45 g/L
Sodium hydrogen phosphate: 3.42 g/L
Potassium dihydrogen phosphate: 2.38 g/L

Production Media (Mineral Salt Media)
Ammonium sulphate: 0.50 g/L
Sodium hydrogen phosphate: 3.80 g/L
Potassium dihydrogen phosphate: 2.65 g/L

Carbon Source
Coconut oil: 20 g/L (obtained from Sigma Aldrich)
Trace element solution: 1 ml/L
Magnesium sulphate heptahydrate: 0.8 g/L

Production: A single colony of Pseudomonas mendocina CH50 was used to inoculate the autoclaved nutrient broth. This nutrient broth was incubated for 16 hours at 30° C. at 150 rpm. Coconut oil was used as the sole carbon source. Autoclaved second stage media (MSM media) was inoculated using the nutrient broth culture as the seed culture. Inoculated second stage media was then incubated at 30° C. at 150 rpm until the optical density reached 1.6 without dilution. This was used as the inoculum to inoculate the production media (MSM media) (10% of the culture volume). The inoculated production media was then incubated for 48 hours at 30° C. at 200 rpm.

Harvesting: The cells were harvested at 48 hours by centrifugation at 4600 rpm for 30 minutes. They were washed thrice, first with distilled water followed by 10% ethanol and then again with distilled water. The cells were homogenized using a homogenizer for approximately 15 minutes. The cells were then kept at −20° C. overnight after which they were placed in a freeze dryer for lyophilisation.

Extraction: Polymer was extracted from the cells using the soxhlet extraction method. The cells are incubated in the soxhlet with methanol for 24 hours at 90° C. under methanol refluxing conditions to remove the impurities. After this, the methanol solution was replaced with the chloroform which was used to extract the polymer from the cells. Extraction was continued using the same batch of cells by incubating them with chloroform for 24 hours under refluxing conditions. This chloroform solution was concentrated using the rotary vacuum evaporator and the polymer was precipitated using ice-cold methanol solution. Yield: 54% dry cell weight, 1.43 g/l.

Characterisation: The resulting polymer was characterised by GC-MS and NMR. The GC-MS results are presented in Table 3:

TABLE 3

| | HO content | HD content | HDD content |
|---|---|---|---|
| | (mol %) | (mol %) | (mol %) |
| Mean | 30.4 | 48.4 | 21.2 |
| SD | 2.1 | 0.8 | 2.0 |

¹H NMR and ¹³C NMR are presented in Figure 2.

Thermal and morphological properties: P(3HO-3HD-3HDD) is a semi-crystalline polymer which is characterized by slow crystallisation. Melting of the crystalline phase was observed only for aged samples. The crystalline phase was not detected by differential scanning calorimetry after melting the polymer in a first heating cycle and cooling the sample at the rate of 20 K/min. The glass transition temperature determined by differential scanning calorimetry was in the range between −42° C. to −45° C. The crystalline phase of P(3HO-3HD-3HDD) melted between 25 to 56° C. The highest melting rate (endothermic peak maximum) was 48±2° C. Enthalpy of fusion (crystallinity degree) changed with polymer storage and for polymer aged at room temperature (for a period of 5 weeks) reached 16±1 J/g.

TABLE 4

| PHA | Tg (° C.) | Tm (° C.) | ΔH (J/g) |
|---|---|---|---|
| P (3HO-co-3HD-co-3HDD) | −42.8 | 48.6 | 11.4 |

Mechanical properties: Film samples were prepared by casting 10 ml of 10 w/v % polymer solution in chloroform into a glass petri dish (6 cm diameter). The samples were left covered at room temperature until solvent evaporation was complete (monitored by weight change). Mechanical properties were determined by tensile testing using samples cut from the solvent-cast film into strips of 5 mm width and 40 mm length. The thickness of the sample was around 300 m. The crosshead speed was 10 mm/min.

P(3HO-3HD-3HDD) is a soft ductile polymer with elongation at break (ε_{U}) 58050%. Young's modulus (E) of P(3HO-3HD-3HDD) was determined as 2.1±0.1 MPa. Ultimate tensile strength (σ_{U}) reached 6.0±1.0 MPa.

Molecular weight (Mw) and polydispersity index (PDI) were determined by GPC. Mw was determined as 333 kDa and PDI as 2.37.

### Example 4—Production of Blends

All PHAs used in the procedure were purified by dissolving in chloroform followed by precipitation with methanol solution. This was repeated several times. The PLLA was a commercially available product (PL38 PURASORB) which was used without further purification.

Blends were produced by dissolving the blend components in a common solvent, chloroform, which is a good solvent for all PHAs and PLLA. Film samples were prepared by solvent casting of polymer solutions.

Polymer solutions were prepared by completely dissolving specified amounts of the first and second blend components in chloroform to achieve a final total polymer concentration of 5 w/v %. The blend solution was kept on the magnetic stirrer to allow mixing for 24 hours at room temperature. 10 ml of the resulting polymer solution was poured into a glass petri dish (6 cm diameter) and left covered at room temperature until solvent evaporation was complete (monitored by weight change).

### Example 5—Testing of Blends

The blends prepared according to Example 4 were subjected to various tests to determine their mechanical properties. As a comparison, the same tests were carried out in respect of the Abbot stent (ABSORB™ BVS) and the known P(3HB)/P(3HO) blends (Basnett et al., Reactive & Functional Polymers 73: 1340-1348, 2013).

Tensile Strength Testing: The following test method was used to determine tensile properties of the blend materials in the form of films (less than 1.0 mm in thickness).

Apparatus:
Device for Measurements of Specimen Geometry: LUJII 150-mm Electronic Digital Caliper or equivalent device accurate and precise to 0.01 mm.
Tensile Tester: Instron Model 5940 Single Column Tabletop Testing System with a 0.5 kN load cell or equivalent.
Gripping Devices: Instron 2710-102 Advanced Screw Side-Action Grips: capacity—500 N. Grip Faces: Rubber coated flat faces (Instron, Cat.: 2702-002)

Materials: Thin films (thinner than 1 mm) of PHA-based materials were prepared by solvent casting. After the films were dried to constant weights, they were packed into non-sealed polyethylene bags and stored at room temperature for 6 weeks.

Method: Preparation of Test Specimens: Cut strips with width of approximately 5 mm from a test film. A circular film disk prepared in a 60 mm Petri dish can be cut into 5 strips with the shortest strips around 36 mm. Cut at least 4 specimens. No specimen shall vary by more than 2% in width along its entire length. Utmost care must be exercised in cutting specimens to prevent nicks and tears along the edges of the specimen that are likely to cause premature failure.

Testing:
Measure and record the thickness of the test specimen to an accuracy of 0.01 mm, at least, in five different places within the gauge length area.
Set the initial gauge length (grip separation) at 23.0 mm and the rate of grip separation at 10.0 mm/min.
Place the specimen in the grips of the testing machine, taking care to align the long axis of the specimen with an imaginary line joining the points of attachment of the grips to the machine. The specimen should be aligned as perfectly as possible with the direction of pull so that no rotary motion that may induce slippage will occur in the grips. Tighten the grips evenly and firmly to the degree necessary to minimize slipping of the specimen during testing.
Start the test and record the load versus extension.
Repeat the testing for the series of specimens prepared.

Fixing a specimen of film sample in the grips of the testing machine always results in a degree of bending of the specimen. This results in an actual length of the sample larger than the set separation between the grips. Therefore the raw load (tensile stress) vs strain curves do not start from “0” separation distance. The initial specimen length is corrected by adding the separation distance, where load starts increasing, to the set distance between the grips. This correction factor is also used for correcting the current specimen deformation by deducing it from the measured separation.

Calculations:

Tensile Strength: Tensile stress (σ) is calculated by dividing the load (F) at a specific time point by the original cross-sectional area (A). The result is expressed in megaPascals (MPa) and reported to three significant figures:

$$σ = \frac{F}{{A}_{0}}$$

The ultimate tensile strength is defined as the maximal value of tensile stress in the stress-strain curve.

Elongation: Percent elongation (ε) is calculated by dividing the corrected distance (lᵢ) of grip separation by the corrected initial length of specimen (l₀) and multiplying by 100:

$$ɛ = \frac{{l}_{i} - {l}_{0}}{{l}_{0}} \times 100 = \frac{Δ l}{{l}_{0}} \times 100$$

Young's Modulus: Young's modulus is calculated as a tangent to the initial linear portion of the stress-strain curve. Obtain the stress-strain curve, select a linear region usually between 0.5 to 1.5% of elongation of the specimen. Use data from this region to calculate a tangent using appropriate software. The result is expressed in gigapascals (GPa) and reported to three significant figures.

Results:

TABLE 5
Mechanical Characterisation of PHA polymer components

| Mechanical | P (3HB) | P (3HO-3HD) | P (3HO-3HD-3HDD) |
|---|---|---|---|
| Properties | Example 1 | Example 2 | Example 3 |
| σ, MPa | 26 | 10.4 | 6 |
| E, GPa | 1.3 | 0.0087 | 0.0021 |
| ε_{b}, % | 2-4 | 580 | 580 |

TABLE 6
Mechanical Characterisation of PHA BLENDS

| Me- | | | P(3HB)/ | | | |
|---|---|---|---|---|---|---|
| chan- | | P(3HB)/ | P(3HO- | | | |
| ical | Abbot | P(3HO- | 3HD- | P(3HB)/ | P(3HB)/ | P(3HB)/ |
| Pro- | Stent | 3HD) | 3HDD) | P(3HO) | P(3HO) | P(3HO) |
| perties | PLLA | 80:20 | 70:30 | 20:80 | 50:50 | 80:20 |
| σ, MPa | 30-50 | 22.4 | 14.7 | 6.8 | 7.8 | 7.4 |
| E, GPa | 1.2-3.0 | 1.44 | 0.92 | 0.028 | 0.078 | 0.085 |
| ε_{b}, % | 2-6 | 26.5 | 19.2 | 58.9 | 22.9 | 10.1 |

TABLE 7
Mechanical Characterisation of PHA BLENDS

| | Abbot | PL38/P (3HO- | PL38/P (3HO- | PL38/P (3HO- |
|---|---|---|---|---|
| Mechanical | Stent | 3HD-3HDD) | 3HD-3HDD) | 3HD-3HDD) |
| Properties | PLLA | 85:15 | 90:10 | 95:5 |
| σ, MPa | 30-50 | 72.1 | 32.6 | 16.7 |
| E, GPa | 1.2-3.0 | 2.9 | 1.2 | 0.9 |
| ε_{b}, % | 2-6 | 11.3 | 47.8 | 2 |

### Example 6—Dip Moulding Preparation of Tubes for Use as Coronary Stents

P(3HB)/P(3HO-3HD) tubes with composition 80/20 were made from polymers by dip molding using polymer solutions in a solvent mixture containing 70 wt. % of highly volatile chloroform and 30 wt. % of low volatile 1,1,2,2-tetrachloroethane.

Solutions of a polymer mixture of P(3HB)/P(3HO-3HD) were prepared by dissolving the required amounts of polymer in the solvent mixture in order to obtain a total polymer concentration of 4 wt. %. Clear solutions were used to form tubes on a cylindrical stainless steel mandrel with diameter 2.3 mm. Tubes were formed by multiple dipping of the mandrel into the polymer solutions. The tube formation was carried out at 50° C. Dipping and withdrawal rate was 200 mm/min. After complete mandrel withdrawal from the polymer solution, 35 sec drying time was used for the first 5 dippings. After this series of dippings, the coated mandrel was kept for partial solvent evaporation for 4 minutes. After that, a next series of 5 dippings was conducted with a drying time of 40 sec between each dipping. After this series of dippings the coated mandrel was kept for partial solvent evaporation for 5 minutes. The next series of 5 dippings was conducted with a drying time of 45 sec and after completion of this series the coated mandrel was kept for partial solvent evaporation for 6 minutes. The last series of five dippings was carried out with a drying time of 50 sec.

The total number of dips was 20 and produced polymeric tubes with a wall thickness of 150 μm. After the last dipping the tubes were left on a mandrel for complete solvent evaporation at 50° C. for 24 hours.

### Example 7—Extrusion of Tubes for Use as Coronary Stents

PLLA/P(3HO-3HD-3HDD) tubes with composition 95/5 were made by extrusion of a pre-mix of the two polymers made via solution. For the preparation of the pre-mix the required amount of polymers were dissolved in chloroform in order to obtain a polymer concentration of 8 wt. %. After complete dissolution, the polymer solution was poured into glass trays and solvent was allowed to evaporate for 3 days at room temperature.

Sheets of the polymer pre-mix were cut into small pieces using a small scale industrial blender. The pre-mix was used for tube extrusion at a barrel temperature of 200° C. to produce tubes with a wall thickness around 200 μm.

### Example 8—Production and Characterisation of Poly(3-Hydroxybutyrate) “P(3HB)” from Walnut Oil by Cupriavidus necator

Producer organism: Cupriavidus necator (formerly known as Ralstonia eutropha)

Production Media
Ammonium chloride: 4 g/L
Disodium hydrogen phosphate. 12 H₂O: 11 g/L
Potassium dihydrogen phosphate: 1.2 g/L (autoclaved at 121° C. for 15 minutes)

Carbon Source
Walnut oil: 20 g/L (autoclaved at 121° C. for 15 minutes) (obtained from Waitrose Ltd.)

Trace element solution: 1 ml/L (filter sterilized)

Magnesium sulphate heptahydrate: 1.4 g/L (autoclaved at 121° C. for 15 minutes)

Production: A single colony of Cupriavidus necator was used to inoculate the autoclaved nutrient broth. This nutrient broth was incubated for 24 hours at 30° C. at 150 rpm. Autoclaved production media was inoculated using the nutrient broth as the seed culture. Inoculated production media was then incubated for 48 hours at 30° C. at 200 rpm.

Harvesting: The cells were harvested at 48 hours by centrifugation at 4600 rpm for 30 minutes. They were washed thrice, first with distilled water followed by 10% ethanol and then again with distilled water. The cells were homogenized using a homogenizer for approximately 15 minutes. The cells were then kept at −20° C. overnight after which they are placed in the freeze dryer for lyophilisation.

Extraction: Polymer was extracted from the cells using soxhlet extraction method. The cells were incubated in the soxhlet with methanol for 24 hours under methanol refluxing conditions as a washing step to remove the impurities. After this, the methanol was replaced with chloroform which was used to extract the polymer from the cells. The cells are incubated with the chloroform for 4 hours under reflux conditions. This chloroform solution was concentrated using the rotary vacuum evaporator and the polymer was precipitated using ice-cold methanol solution. Extraction was continued using the same batch of cells by incubating them twice with chloroform solution at 70° C. for 24 hours. This chloroform solution was again concentrated using the rotary vacuum evaporator and the polymer was precipitated using ice-cold methanol solution. Yield: 50% dry cell weight, 1.97 g/l.

Characterisation: The resulting polymer was characterised by GC-MS and NMR. ¹H NMR and ¹³C NMR are presented in FIG. 3.

Thermal and morphological properties: P(3HB) is a semi-crystalline polymer. Glass transition temperature determined by differential scanning calorimetry was in the range between 0° C. to 3° C. The crystalline phase of P(3HB) melted in the wide temperature range between 120 to 175° C. The highest melting rate (endothermic peak maximum) was 171±2° C. Enthalpy of fusion (crystallinity degree) changed with polymer storage and for polymer aged at room temperature reached 88±2 J/g.

TABLE 8

| PHA | Tg (° C.) | Tm (° C.) | ΔH (J/g) |
|---|---|---|---|
| P (3HB) | 2.9 | 168.4 | 71.4 |

Mechanical properties: Film samples were prepared by casting 10 ml of 10 w/v % polymer solution in chloroform into a glass petri dish (6 cm diameter). The samples were left covered at room temperature until solvent evaporation was complete (monitored by weight change). Mechanical properties were determined by tensile testing using samples cut from the solvent-cast film into the strips of 5-mm width and 40-mm long. The thickness of the sample was around 200 m. The crosshead speed was 5 mm/min.

P(3HB) is a rigid polymer with elongation at break (ε_{U}) 24%. Young's modulus (E) of P(3HB) was 1.3±0.2 GPa. Ultimate tensile strength (σ_{U}) reached 262 MPa.

Molecular weight (Mw) and polydispersity index (PDI) were determined by GPC. Mw was determined as 606 kDa and PDI as 2.5.

The P(3HB) polymer may be used as the second component in a blend according to the invention.

### Example 9—Production and Characterisation of Poly (3-Hydroxyoctanoate-Co-3-Hydroxydecanoate) “P(3HO-3HD)”—23 Mol % HO, 77 Mol % HD

Producer organism: Pseudomonas mendocina CH50

Media Composition:

Second stage (Mineral salt medium)
Ammonium sulphate: 0.45 g/L
Sodium hydrogen phosphate: 3.42 g/L
Potassium dihydrogen phosphate: 2.38 g/L (autoclaved at 121° C. for 15 minutes)

Production Media (Mineral Salt Media)
Ammonium sulphate: 0.50 g/L
Sodium hydrogen phosphate: 3.80 g/L
Potassium dihydrogen phosphate: 2.65 g/L (autoclaved at 121° C. for 15 minutes)

Carbon Source
Sugarcane molasses: 20 g/L (bought from Holland and Barret) (autoclaved at 110° C. for 10 minutes)

Trace element solution: 1 ml/L (filter sterilized)
Magnesium sulphate heptahydrate: 0.8 g/L (autoclaved at 121° C. for 15 minutes)

Production: A single colony of Pseudomonas mendocina CH50 was used to inoculate the autoclaved nutrient broth. This nutrient broth was incubated for 16 hours at 30° C. at 150 rpm. Autoclaved second stage media was inoculated using the nutrient broth culture as the seed culture. Inoculated second stage media was then incubated at 30° C. at 150 rpm until the optical density reached 1.6 without dilution. This was used as the inoculum to inoculate the production media (10% culture volume). Inoculated production media was then incubated for 48 hours at 30° C. at 150 rpm.

Harvesting: The cells were harvested at 48 hours by centrifugation at 4600 rpm for 30 minutes. They were washed thrice, first with distilled water followed by 10% ethanol and then again with distilled water. The cells were homogenized using a homogenizer for approximately 15 minutes. The cells were then kept at −20° C. overnight after which they are placed in the freeze dryer for lyophilisation.

Extraction: Polymer was extracted from the cells using soxhlet extraction method. The cells were incubated in the soxhlet with methanol for 24 hours under reflux conditions as a washing step to remove the impurities. After this, the methanol was replaced with chloroform which was used to extract the polymer from the cells. Extraction was carried out by incubating the cells with chloroform solution for 24 hours under reflux conditions. This chloroform solution was concentrated using the rotary vacuum evaporator and the polymer was precipitated using ice-cold methanol solution. Yield: 37.5% dry cell weight, 0.46 g/l.

Characterisation: The resulting polymer was characterised by GC-MS and NMR. ¹H NMR and ¹³C NMR are presented in FIG. 4.

Thermal and morphological properties: P(3HO-3HD) is a semi-crystalline polymer which is characterized by slow crystallization. Melting of the crystalline phase was observed only for aged samples. The crystalline phase was not detected by differential scanning calorimetry after melting the polymer in first heating cycle and cooling the sample at the rate of 20 K/min. Glass transition temperature determined by differential scanning calorimetry was in the range between −41° C. to −46° C. The crystalline phase of P(3HO-3HD) melted between 28 to 70° C. The highest melting rate (endothermic peak maximum) was 53±2° C. Enthalpy of fusion (crystallinity degree) changed with polymer storage and for polymer aged at room temperature reached 21±2 J/g.

TABLE 9

| PHA | Tg (° C.) | Tm (° C.) | ΔH (J/g) |
|---|---|---|---|
| P (3HO-co-3HD) | −41.5 | 53.0 | 21.0 |

Mechanical properties: Film samples were prepared by casting 10 ml of 10 w/v % polymer solution in chloroform into a glass petri dish (6 cm diameter). The samples were left covered at room temperature until solvent evaporation was complete (monitored by weight change). Mechanical properties were determined by tensile testing using samples cut from the solvent-cast film into the strips of 5-mm width and 40-mm long. The thickness of the sample was around 300 m. The crosshead speed was 10 mm/min.

P(3HO-3HD) is a soft ductile polymer with elongation at break (ε_{U}) 635±25%. Young's modulus (E) of P(3HO-3HD) was 11.4±0.3 MPa. Ultimate tensile strength (σ_{U}) reached 15.1±0.3 MPa.

Molecular weight (Mw) and polydispersity index (PDI) were determined by GPC. Mw was determined as 449.3 kDa and PDI as 1.5.

The P(3HO-3HD) polymer may be used as the first component in a blend according to the invention.

1. A stent having a stent body which comprises a polymer blend comprising:
(a) from 5 to 40 wt. % of a first component which is a PHA copolymer comprising two or more different medium chain length hydroxyalkanoate monomer units; and
(b) from 60 to 95 wt. % of a second component which is either a PHA homopolymer containing a short chain length hydroxyalkanoate monomer unit, or a polylactide (PLA).

2. A stent as claimed in claim 1, wherein the first component is a binary or ternary PHA copolymer.

3. A stent as claimed in claim 1 or claim 2, wherein the PHA copolymer comprises hydroxyalkanoate monomer units which, independently of one another, contain 6 or more carbon atoms, preferably from 6 to 16 carbon atoms.

4. A stent as claimed in claim 3, wherein the PHA copolymer comprises hydroxyalkanoate monomer units which, independently of one another, contain 8, 10 or 12 carbon atoms.

5. A stent as claimed in any one of the preceding claims, wherein the PHA copolymer comprises hydroxyalkanoate units which are independently selected from 3-hydroxy and 4-hydroxyalkanoates.

6. A stent as claimed in claim 5, wherein each hydroxyalkanoate unit is a medium chain length 3-hydroxyalkanoate.

7. A stent as claimed in claim 6, wherein each hydroxyalkanoate unit is independently selected from the group consisting of 3-hydroxyoctanoate (3HO), 3-hydroxydecanoate (3HD) and 3-hydroxydodecanoate (3HDD).

8. A stent as claimed in claim 1, wherein the PHA copolymer is poly(3-hydroxyoctanoate-co-3-hydroxydecanoate) or poly(3-hydroxyoctanoate-co-3-hydroxydecanoate-co-3-hydroxydodecanoate).

9. A stent as claimed in any one of the preceding claims, wherein the chirality of the hydroxy-substituted carbon atom in each hydroxyalkanoate unit in the PHA copolymer is of the R-configuration.

10. A stent as claimed in any one of the preceding claims, wherein the first component is a binary PHA copolymer which contains 3-hydroxydecanoate (3-HD) monomer units in an amount ranging from 60 mol % to 85 mol % (based on the weight average molecular weight, Mw, of the PHA copolymer) and/or 3-hydroxyoctanoate monomer units in an amount from 20 to 35 mol % (based on the weight average molecular weight, Mw, of the PHA copolymer).

11. A stent as claimed in any one of claims 1 to 9, wherein the first component is a ternary PHA copolymer which contains 3-hydroxydecanoate (3-HD) monomer units in an amount ranging from 40 mol % to 60 mol % (based on the weight average molecular weight, Mw, of the PHA copolymer) and/or 3-hydroxyoctanoate monomer units in an amount from 20 to 40 mol % (based on the weight average molecular weight, Mw, of the PHA copolymer).

12. A stent as claimed in any one of the preceding claims, wherein the first component is a PHA copolymer which contains 3-hydroxydodecanoate (3-HDD) monomer units in an amount ranging from 10 to 30 mol % (based on the weight average molecular weight, Mw, of the PHA copolymer).

13. A stent as claimed in any one of the preceding claims, wherein the PHA copolymer has a molecular weight in the range from 50 to 600 kDa.

14. A stent as claimed in any one of the preceding claims, wherein the PHA copolymer is obtained or obtainable by culturing of a microorganism selected from Pseudomonas putida, Pseudomonas oleovorans, Pseudomonas mendocina CH50, Pseudomonas fluorescence, Pseudomonas aeruginosa, Pseudomonas raguenesii, Pseudomonas guezennei, Pseudomonas stutzeri, Pseudomonas cepacia, and Comamonas testosteronii.

15. A stent as claimed in claim 14, wherein said microorganism is Pseudomonas mendocina CH50.

16. A stent as claimed in claim 14 or claim 15, wherein said microorganism is grown in a culture medium which comprises glucose or coconut oil as a carbon source.

17. A stent as claimed in any one of the preceding claims, wherein the second component of the polymer blend is a PHA homopolymer containing a short chain length hydroxyalkanoate monomer unit.

18. A stent as claimed in claim 17, wherein the PHA homopolymer comprises hydroxyalkanoate monomer units which each contain 3, 4 or 5 carbon atoms.

19. A stent as claimed in claim 18, wherein the PHA homopolymer is poly(3-hydroxybutyrate).

20. A stent as claimed in any one of the preceding claims, wherein the PHA homopolymer has a molecular weight in the range from 200 kDa to 2 MDa.

21. A stent as claimed in any one of the preceding claims, wherein the PHA homopolymer is obtained or obtainable by culturing of a microorganism selected from Cupriavidus necator, Alcaligenes latus, Bacillus cereus, Aeromonas caviae, Rhodospirillum rubrum, Methylobacterium extorquens, Halomonas boliviensis LC 1, Bacillus subtilis, and Bacillus megaterium.

22. A stent as claimed in claim 21, wherein said microorganism is Bacillus subtilis OK2.

23. A stent as claimed in claim 21 or claim 22, wherein said microorganism is grown in the presence of a culture medium which comprises glucose as a carbon source.

24. A stent as claimed in any one of claims 1 to 16, wherein the second component of the polymer blend is a polylactide (PLA).

25. A stent as claimed in claim 24, wherein the second component of the polymer blend is poly(L-lactic acid).

26. A stent as claimed in any one of the preceding claims, wherein the polymer blend is selected from one of the following:
P(3HO-3HD)/P(3HB)
P(3HO-3HD-3HDD)/P(3HB)
P(3HO-3HD-3HDD)/PLA
P(3HO-3HD)/PLA
P(3HO-3HD-3HDD)/PLLA
P(3HO-3HD)/PLLA
P(3HO-3HD-3HDD)/PDLA
P(3HO-3HD)/PDLA

27. A stent as claimed in any one of the preceding claims, wherein the first component is present in an amount in the range from 20 to 30 wt. % (based on the total weight of the blend).

28. A stent as claimed in any one of the preceding claims, wherein the second component is a PHA homopolymer which is present in an amount in the range from 70 to 80 wt. % (based on the total weight of the blend).

29. A stent as claimed in any one of claims 1 to 27, wherein the second component is a polylactide which is present in an amount in the range from 80 to 90 wt. % (based on the total weight of the blend).

30. A method of producing a stent as claimed in any one of claims 1 to 29, said method comprising forming a stent body from a polymer blend which comprises:
(a) from 5 to 40 wt. % of a first component which is a PHA copolymer comprising two or more different medium chain length hydroxyalkanoate monomer units; and
(b) from 60 to 95 wt. % of a second component which is either a PHA homopolymer containing a short chain length hydroxyalkanoate monomer unit, or a polylactide (PLA).

31. A polymer blend comprising:
(a) from 5 to 40 wt. % of a first component which is a PHA copolymer comprising two or more different medium chain length hydroxyalkanoate monomer units; and
(b) from 60 to 95 wt. % of a second component which is either a PHA homopolymer containing a short chain length hydroxyalkanoate monomer unit, or a polylactide (PLA)

32. A polymer blend as claimed in claim 31, wherein said first and second components are as defined in any one of claims 2 to 29.

33. A polymer blend as claimed in claim 31 or claim 32 having one or more of the following mechanical properties: a tensile strength (σ) greater than 20 MPa; a Young's modulus (E) greater than 1 GPa; and an elongation at break (ε_{b}) of at least 10%.

34. A method of producing a PHA copolymer, said method comprising the steps of:
(a) culturing Pseudomonas mendocina CH50 in a culture medium comprising a carbon source other than glucose;
(b) harvesting biomass from the culture medium;
(c) extracting PHA from the harvested biomass; and
(d) optionally purifying the crude PHA whereby to obtain a purified PHA.

35. A PHA copolymer obtained or obtainable by culturing Pseudomonas mendocina CH50 in the presence of a culture medium which comprises a carbon source other than glucose.
